(19) United States
(12) Patent Application Publication
(10) Pub. No.: US 2026/0165328 A1
(43) Pub. Date: Jun. 18, 2026

(54) STRAIN OF BACILLUS VELEZENSIS AND ITS USE

(71) Applicant: BEIJING ACADEMY OF AGRICULTURE AND FORESTRY SCIENCES, Beijing (CN)

(72) Inventors: Wei ZHANG, Beijing (CN); Jiye YAN, Beijing (CN); Mei LIU, Beijing (CN); Xinghong LI, Beijing (CN); Yonghua LI, Beijing (CN); Hui WANG, Beijing (CN)

(21) Appl. No.: 19/114,684

(22) PCT Filed: May 10, 2023
(86) PCT No.: PCT/CN2023/093264

§ 371 Date: Mar. 3, 2026

(30) Foreign Application Priority Data
May 2, 2023 (CN) 202310482724.3

(51) Int. Cl. A01N 63/22; A01N 25/14; A01P 3/00; C12N 1/205; C12R 1/07

(52) U.S. Cl. CPC A01N 63/22; A01N 25/14; A01P 3/00; C12N 1/205; C12R 2001/07

(57) ABSTRACT

The present disclosure provides a strain of Bacillus velezensis and its use. The strain of Bacillus velezensis BJ-1 disclosed herein has a depository number CGMCC No. 24113. This strain exhibits antibacterial activity against plant pathogenic fungi and possesses broad-spectrum antibacterial effect. The result of in vitro leaf inoculation detection showed that the fermentation liquid derived from Bacillus velezensis BJ-1 exhibited significant prevention and control effects on tomato gray mold. The relative prevention and control effects of 1×10⁷ CFU/mL the fermentation liquid on tomato gray mold were determined to be 68.95% and 88.28%, respectively. The result of the field trial demonstrated that Bacillus velezensis BJ-1 water-dispersible powder exhibited significant prevention and control effects on tomato gray mold. The relative prevention and control effects of 2.0×10¹⁰ CFU/g (450 g/mu) Bacillus velezensis water-dispersible powder were determined to be 80.48%. The strain of Bacillus velezensis disclosed herein has potential for biocontrol, including the manufacturing biocontrol agents and microbial fertilizers for plant pathogenic fungi.

### FIELD OF THE INVENTION

The present disclosure belongs to the field of microbial application, in particular, relates to a strain of Bacillus velezensis and a its use in controlling plant diseases such as tomato gray mold.

### BACKGROUND OF THE INVENTION

The hosts of Botrytis cinerea are diverse. Botrytis cinerea can infect fruits and vegetables such as grapes, strawberries, cherries, tomatoes, and watermelons, leading to gray mold in crops worldwide. Botrytis cinerea generally causes a decrease in yield by 10%˜20% and up to 60% or more in severe cases. The chemical prevention and control measures are crucial for preventing and controlling gray mold in crops in current actual production (Kang Lijuan, Zhang Xiaofeng, Wang Wenqiao, et al. Determination of resistance and fitness of grey mold. Chinese Journal of Agricultural Sciences, 2003,2:39-42). However, the rapid reproduction, high genetic variation, and high fitness of Botrytis cinerea contribute to its rapid development of resistance to chemical pesticides, resulting in a decrease in the effectiveness of prevention and control (Hahn M. The rising threat of fungicide resistance in plant pathogenic fungi: Botrytis as a case study. Journal of Chemical Biology, 2014, 7(4): 133-141). In addition, the long-term, repeated, and extensive utilization of chemical pesticides can lead to soil, water and atmospheric pollution, thereby disrupting the ecological balance. Biocontrol agents have low toxicity, are environmentally friendly and safe, and align with the principles of sustainable development (Kaur S, Samota M K, Choudhary M, et al. How do plants defend themselves against pathogens-Biochemical mechanisms and genetic interventions. Physiology and Molecular Biology of Plants, 2022, 28(2): 485-504). Therefore, it is urgent to develop biocontrol agents for gray mold in crops. Bacillus spp. exists extensively in nature, and the dormant spores from Bacillus spp. under adverse conditions not only have a significantly low water content, but also exhibits resistances to acidity, salinity, high temperatures, and extrusion. The metabolism of the dormant spores can also generate ten kinds of antibacterial substances including various enzymes, bacteriocin, cyclolipids, and bacteriophage-like particles. Bacillus spp., in comparison to other beneficial microorganisms, exhibits a significantly higher survival rate, robust reproductive capacity, exceptional environmental adaptability and resistance. Consequently, it stands as the most extensively utilized biocontrol bacterium in biocontrol agent products (Ivica Dimkiki I, Janakiev T, Petroviki M, et al. Plant-associated Bacillus and Pseudomonas antimicrobial activities in plant diseases suppression via biological control mechanisms-A review, Physiological and Molecular Plant Pathology, 2022, 117:101754). Bacillus spp. comprises numerous species, among which the most frequently employed biocontrol agents include Bacillus subtilis and Bacillus amyloliquefaciens, Paenibacilluspolmyxa, Bacillus cereus, Bacillus licheniformis and so on. In recent years, Bacillus velezensis has garnered significant attention from researchers due to its potent antibacterial efficacy. Numerous studies have demonstrated that Bacillus velezensis plays an important role in the prevention and control of lettuce root rot caused by Rhizoctonia solani and tomato bacterial wilt caused by Ralstonia solanacearum (Cao Y, Pi H L, Chandrangsu P, et al. Antagonism of two plant-growth promoting Bacillus velezensis isolates against Ralstonia solanacearum and Fusarium oxysporum. Scientific Reports, 2018,8(1): 4360). The field prevention and control effects of Bacillus velezensis BS87 and RK1 on strawberry wilt disease are comparable to those of copper hydroxide (Nam M H, Park M S, Kim H G, et al. Biological control of strawberry Fusarium wilt caused by Fusarium oxysporumf.spfragariae using Bacillus velezensis BS87 and RK1 formulation. Journal of Microbiology and Biotechnology, 2009,19(5): 520-524). The prevention and control effects of Bacillus velezensis-83, isolated from mango leaves by Balderas et al., on mango anthracnose is comparable to those of chemical treatments (Balderas-Ruiz K A, Bustos P, Santamaria R I, et al. Bacillus velezensis 83 a bacterial strain from mango phyllosphere, useful for biological control and plant growth promotion.AMB Express, 2020,101:7-10.)

### SUMMARY OF THE INVENTION

In order to solve the above problems, the present disclosure provides a strain of Bacillus velezensis and its use.

The strain of Bacillus velezensis disclosed herein is classified as Bacillus velezensis, isolated from grapevine trunk tissues affected by Grapevine Trunk Diseases in Huailai County, Zhangjiakou, Hebei Province, and named Bacillus velezensis BJ-1, which has been deposited in China General Microbiological Culture Collection Center (Address: No. 3, Courtyard No. 1, Beichenxi Road, Chaoyang District, Beijing) on Dec. 16, 2021, with a depository number CGMCC No. 24113.

The strain of Bacillus velezensis disclosed herein does not produce a pigment on LB medium, and the colony appears opalescent and opaque, round or nearly round, with non-smooth edges and obvious wrinkles on its surface. Bacillus velezensis BJ-1 exhibits physiological and biochemical characteristics including Gram staining positive and produces cellulase, “but it does not produce protease and amylase.

The strain of Bacillus velezensis disclosed herein exhibits antibacterial activity against plant pathogenic fungi and possesses a broad-spectrum antibacterial effect.

The plant pathogenic fungi are one or more of Lasiodiplodia theobromae, Botryosphaeria dothidea, Neofusicoccum parvum, Diaporthe sojae, Diaporthe eres, Diaporthe honkonggensis, Botrytis cinerea, Colletotrichum viniferum, Colletotrichum sojae, Colletotrichum aenigma, Colletotrichum gloeosporioides, Colletotrichum fructicola, Colletotrichum siamense, Colletotrichum acutatum, Neopestalotiopsis sp., Neopestalotiopsis Rosae, Coniella vitis, Dactylonectria alcacerensis, Dactylonectria macrodidyma, Lasiodiplodiapseudotheobromae, Dactylonectria torresensis, Fusarium oxysporum, anastomosis group A of binucleate Rhizoctonia, anastomosis group G of binucleate Rhizoctonia, Alternaria alternata, Cladosporium cladosporioides, Sclerotinia sclerotiorum. The inhibition rates of mycelium growth for 59 strains derived from 26 plant pathogenic fungi range from 53.76% to 98.45%. These strains exhibit promising potential for the development of biologics for plant fungal diseases.

The present disclosure also provides a biopesticide for plant diseases, wherein the active ingredient of the biopesticide is the strain of Bacillus velezensis.

The biopesticide also includes excipients and/or auxiliaries acceptable to the pesticide formulation.

In a preferred embodiment, the biopesticide is the form of water-dispersible powders containing 1.0×10⁸ CFU/g or more of Bacillus velezensis BJ-1; and Bacillus velezensis BJ-1 is Bacillus velezensis defined in claim 1.

Preferably, the water-dispersible powders are composed of the following components:

Bacillus velezensis BJ-1 at a final concentration of 1.0×10⁸ to 1.0×10¹¹ CFU/mL, preferably 2.0×10¹⁰ CFU/mL; 6.0% polyvinyl alcohol; 4.0% sodium dodecyl sulfate; 4% ammonium sulfate; 4% polyethylene glycol; and talc powder in the remaining percentage content. Herein, the percentage content refers to a mass percentage.

The strain of Bacillus velezensis disclosed herein exhibits effective prevention and control effects on tomato gray mold caused by Botrytis cinerea both indoors and in the field. The results of the in vitro leaf inoculation detection showed that the fermentation liquid derived from Bacillus velezensis BJ-1 exhibit significant prevention and control effects on tomato gray mold. Specifically, the relative prevention and control effects of 1×10⁷ CFU/mL of the fermentation liquid on tomato gray mold were determined to be 68.95% and 88.28%, respectively. The results of the field trials demonstrate that the water-dispersible powders of Bacillus velezensis BJ-1 exhibit significant prevention and control effects on tomato gray mold. The relative prevention and control effects of 2.0×10¹⁰ CFU/g (450 g/mu) Bacillus velezensis water-dispersible powder are found to be 80.48%. The present invention has potential for biocontrol, including the manufacture of biocontrol agents and microbial fertilizers for plant pathogenic fungi.

The strain of Bacillus velezensis disclosed herein exhibits preferable prevention and control effects on various plant pathogenic fungi and demonstrates effective prevention and control effects on tomato gray mold caused by Botrytis cinerea in the field. Therefore, it holds promising application prospects for the prevention and control of tomato gray mold in fruits and vegetables caused by Botrytis cinerea. Moreover, the strain is suitable for industrial production due to simple culture conditions, rapid growth, and easy preservation.

### BRIEF DESCRIPTION OF THE DRAWINGS

FIG. 1 shows the colony morphology and scanning electron microscope (SEM) image of the strain BJ-1.

FIG. 2 shows the results of amylase detection for the strain BJ-1,
Top: Escherichia coli JM109; Lower left: Bacillus subtilis IPEP6; Lower right: strain BJ-1.

FIG. 3 shows the results of proteinase detection for the strain BJ-1,
Top: Escherichia coli JM109; Lower left: Bacillus subtilis IPEP6; Lower right: strain BJ-1.

FIG. 4 shows the results of cellulase detection for the strain BJ-1;
Top: Escherichia coli JM109; Lower left: Bacillus subtilis IPEP6; Lower right: strain BJ-1.

FIG. 5 shows the results of lipase detection for the strain BJ-1;
Top: Escherichia coli JM109; Lower left: Bacillus subtilis IPEP6; Lower right: strain BJ-1.

FIG. 6 shows the results of glucose fermentation detection for the strain BJ-1;
Top: Escherichia coli JM109; Lower left: strain BJ-1; Lower right: Bacillus subtilis IPEP6.

FIG. 7 shows the results of sucrose fermentation detection for the strain BJ-1;
Top: Escherichia coli JM109; Lower left: strain BJ-1; Lower right: Bacillus subtilis IPEP6.

FIG. 8 shows the results of lactose fermentation detection for the strain BJ-1;
Top: Escherichia coli JM109; Lower left: strain BJ-1; Lower right: Bacillus subtilis IPEP6.

FIG. 9 shows the results of mannitol fermentation detection for the strain BJ-1;
Top: Escherichia coli JM109; Lower left: strain BJ-1; Lower right: Bacillus subtilis IPEP6.

FIG. 10 shows the results of inositol fermentation detection for the strain BJ-1;
Top: Escherichia coli JM109; Lower left: strain BJ-1; Lower right: Bacillus subtilis IPEP6.

FIG. 11 shows the results of Gram straining method of the strain BJ-1;

FIG. 12 shows the phylogenetic tree based on the 16S rDNA.

### DETAILED DESCRIPTION OF EMBODIMENTS

### Deposit of Biological Materials

Strain Name: Bacillus velezensis BJ-1
Classification: Bacillus velezensis
Depository Date: Dec. 16, 2021
Depository Authority: China General Microbiological Culture Collection Center (Address: No. 3, No. 1, Beichenxi Road, Chaoyang District, Beijing)
Depository Number: CGMCC No. 24113.

### Example 1. Acquisition and Identification of Bacillus velezensis BJ-1

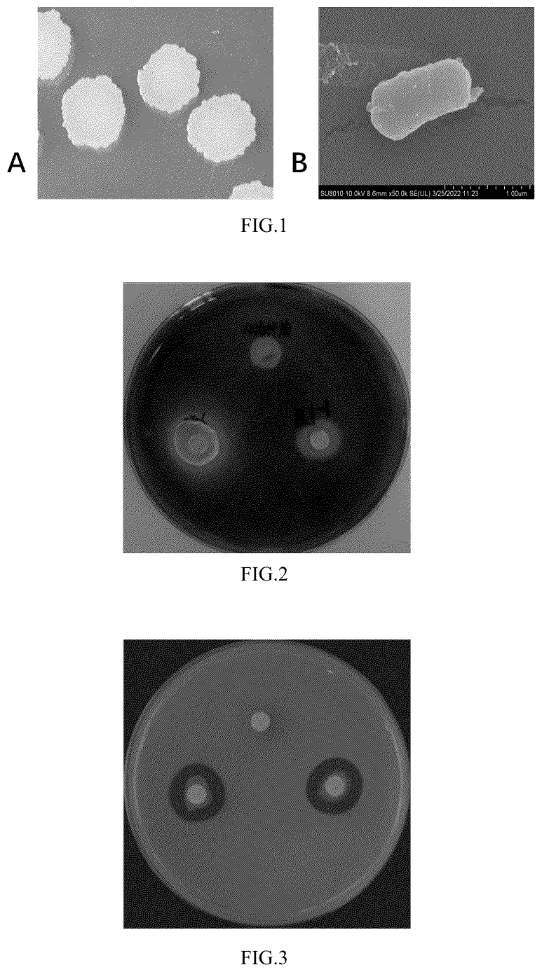

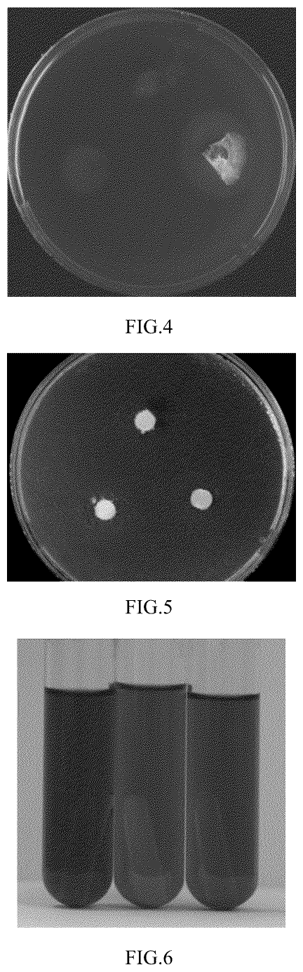

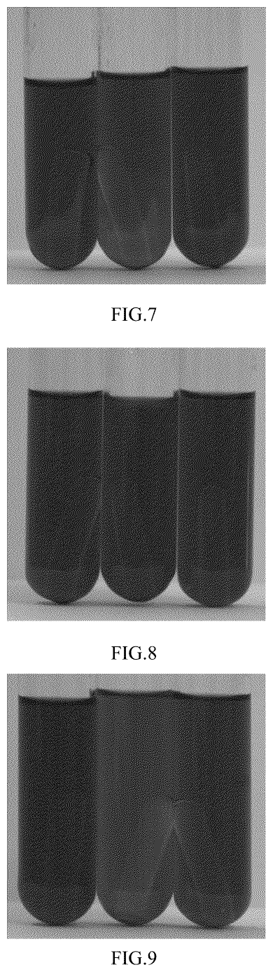

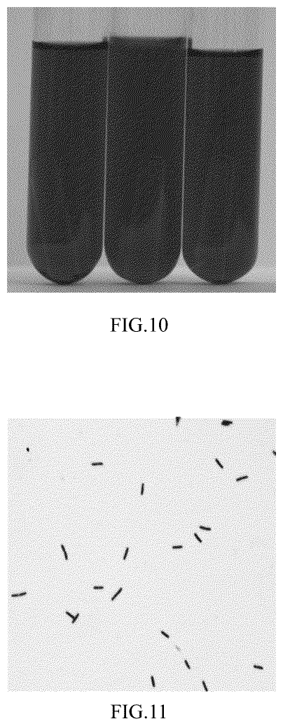

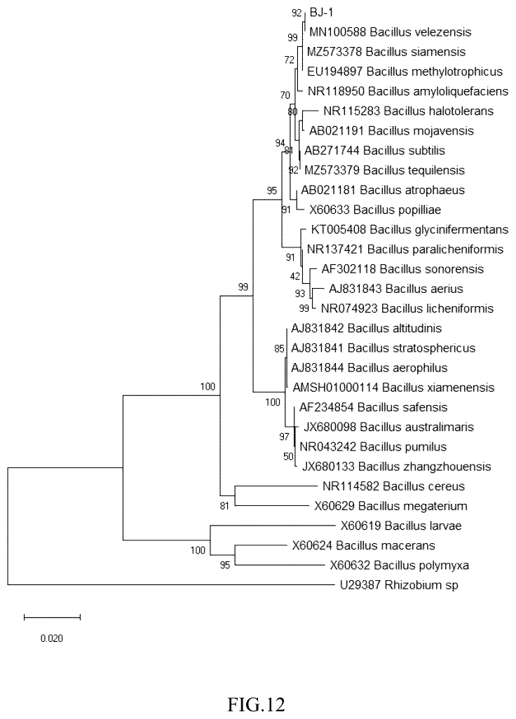

The pathogenic fungi were isolated from grapevine trunk tissues affected by grapevine trunk diseases collected from grapevines in Haidian District, Beijing by using a tissue isolation method, comprising: 1) observing and recording the symptoms associated with grapevine trunk diseases; 2) cutting sections with a length of 2 cm at intervals of 10 cm from grapevine trunks with varying degrees of infection, removing the bark from sections with grapevine trunk diseases and extracting 5 mm² tissue blocks from the junction between healthy and infected tissues, disinfecting these tissue blocks using a solution of 2% sodium hypochlorite for 2 minutes, followed by 70% ethanol for 30 seconds, washing them three times with sterile water, placing the tissue blocks on sterile filter paper to dry completely, transferring them onto PDA plates with a density of approximately 4-5 tissue blocks per plate, and sealing the plates using parafilm; 3) incubating the plates at a temperature of 25° C. in darkness for three days, observing and recording colony growth and calculating the corresponding isolation ratio after three days; and 4) collecting a small amount of hyphae from the edges of colonies grown on a new PDA plate or other suitable medium that induces spore production effectively, and isolating monospores after conidia are generated successfully. During isolation of pathogenic fungi, it was found that a strain exhibited a good antagonistic effect against the pathogenic fungi. The strain was subsequently streaked on LB agar plates for purification, thereby acquiring a pure culture of the strain. Upon identification, the strain exhibiting antagonistic activity against the pathogenic fungi was designated as BJ-1. The colony morphology of the strain BJ-1 is shown in FIG. 1. The colony of the strain BJ-1 appears round or nearly round, with an off-white coloration, an irregular periphery, and a slightly wrinkled surface.

### Physiological and Biochemical Characteristics of the Strain BJ-1

### 1. Physiological and Biochemical Characterization of the Strain

### 1.1. Amylase Detection

Detection medium: A starch-selective culture medium (LB culture medium+1.0% soluble starch): 10 g peptone, 5 g yeast extract, 5 g NaCl, 10 g soluble starch, 20 g agar, and 1,000 mL distilled water were mixed to prepare the detection medium, with the pH adjusted to 7.8.
Detection method: The detection plates were inoculated with 10 μl of the vigorously shaken and cultured cultures containing the strain to be detected (the target strain) or control strains without amylolytic enzyme activity (Escherichia coli and Bacillus subtilis). After air-drying, the plates were incubated at a constant temperature of 28° C. for a period of 24 to 48 hours and dyed by covering the plates with diluted iodine solution followed by rinsing with water. The sizes of the transparent zones formed around each colony were observed.
Detection result: As depicted in FIG. 2, it is evident that there were distinct transparent zones around the target strain BJ-1 on the detection plates, indicating that the strain can produce amylase and exhibited a positive starch hydrolysis. There was no transparent zone around the control strain E. coli, indicating the absence of amylase activity.

### 1.2. Protease Detection

Detection Plate: 10% of skim-milk-agar medium (1 g agar powder, /100 mL water). Sanyuan skim-milk, which was first heated in the microwave to remove any fat film, was used in the plate. Using a pipette, 10 mL of the treated Sanyuan skim-milk was aspirated and then added to 100 mL of sterilized and cooled agar-water medium.
Detection Method: The tested strain was cultured in NA medium overnight. 10 μl of the culture was spotted onto the detection plate, and then incubated at a constant temperature of 28° C. After 12 hours, protein degradation zones were observed.
Detection results: As shown in FIG. 3, it can be seen that there was distinct transparent zones around the target strain BJ-1, indicating that the strain can produce protease, with a positive hydrolysis result. There was no transparent zone around the control strain Escherichia coli, indicating no protease activity.

### 1.3. Cellulase Detection

Detection Plate: 0.2% carboxymethyl cellulose sodium was added to Half-LB medium (containing half of the carbon source and half of the nitrogen source).
Detection Method: the tested strain and the target strain were co-cultured in NA medium overnight. 10 μl of the culture was spotted onto the detection plate, incubated at 28° C. for 24 hours, washed, and stained with 1 g/L Congo red solution for 1 hour, decolorized by immersing in 1 M NaCl solution, followed by two washes of 30 minutes each, and observation for presence of cellulose hydrolysis zones around the colonies.
Detection Results: As shown in FIG. 4, it can be seen that there were white transparent zones around the target strain BJ-1, indicating that the strain can produce cellulase, with cellulase hydrolysis being positive. There was no transparent zone around the control strain E. coli, indicating a lack of cellulase hydrolytic enzyme activity.

### 1.4. Lipase Detection

Detection Medium: The lipid-medium contained 10 g peptone, 5 g beef extract, 10 g peanut oil or sesame oil, 1 mL of 1.6% neutral-red solution, 15 g agar, and 1000 mL of distilled water, pH 7.2.
Detection Method: the lipid-medium was prepared and sterilized. Before pouring into a plate, the lipid-medium was shaken thoroughly, then poured into the petri dish to ensure uniform distribution of oils. The tested strain was inoculated onto the solidified plate, air-dried, and incubated at 28° C. for 24 hours. The colonies on the plate were observed. If red spots appear, it indicates that the fatty acids have been hydrolyzed, which is a positive reaction.
Detection Results: As shown in FIG. 5, it can be seen that there were no red spot around the colonies of all tested strains, indicating that they cannot produce lipase, with negative lipase hydrolysis.

### 1.5. Fermentation Experiments of Sugar and Alcohol

### Basal Medium for Sugar Fermentation:

10 g peptone, 5 g NaCl, 1000 ml distilled water and 1.6% solution of bromocresol purple in ethanol, pH adjusted to 7.6.

### Detection Method:

The basal medium for sugar fermentation was supplemented with glucose (1%), lactose (0.75%), sucrose (0.75%), mannitol (0.75%), and inositol (0.75%), separately. The sugar-containing basal medium was dispensed into the test tubes, each of which included a Durham fermentation tube, the tested strain were inoculated into the sugar-containing basal medium and incubated at 30° C. for 7 days. The uninoculated basal medium was used as a control. Changes in color in each of the tested tubes and the presence of gas bubbles in the Durham fermentation tubes were observed. The indicator of bromothymol blue had a pH range from 5.2 (yellow) to 6.8 (purple).

### Detection Results:

The results of the glucose fermentation experiment are shown in FIG. 6. From the FIGURE, it can be seen that the medium colors in the tested tubes of strain BJ-1 changed from purple to yellow, and no bubbles were produced in the Durham tubes, indicating that the tested strain can ferment glucose to produce acid but not gas.

The results of the sucrose fermentation experiment are shown in FIG. 7. From the FIGURE, it can be seen that the medium colors in the tested tubes of the strain BJ-1 changed from purple to yellow, and no bubbles were produced in the Durham tube, indicating that the tested strain can ferment sucrose to produce acid but not gas.

The results of lactose fermentation experiment are shown in FIG. 8. From the FIGURE, it can be seen that there was no change in the color of the culture tubes for the strain BJ-1, and no bubbles were produced in the Durham tubes, indicating that the strain BJ-1 cannot decompose lactose.

The results of mannitol fermentation experiment are shown in FIG. 9. From the FIGURE, it can be seen that the colors of the tested tubes for the strain BJ-1 changed from purple to yellow, but not gas bubble was produced in the Durham tubes, indicating that the tested strain can decompose mannitol to produce acid but not gas.

The results of inositol fermentation experiment are shown in FIG. 10. From the FIGURE, it can be seen that there was no change in the color of the test tube culture medium of strain BJ-1, and no bubbles were produced in the Durham tube, indicating that the tested strain cannot decompose inositol.

### 2. Gram Staining of Strain

### 2.1 Materials and Methods

Staining Agents and Reagents: crystal violet, Lugol's iodine solution, 95% alcohol, and safranin.

Staining Method: The tested strain and the target strains were cultured on LB agar plates for 48 hours. A clean slide was taken. Two drops of distilled water were separately added to the left and right sides of the slide. The strains were taken and inoculated on the slide using aseptic techniques. The tested strain was inoculated on the left side, and the target strains were inoculated on the right side, forming concentrated bacterial suspensions. Another clean slide was taken and 1-2 loops of the fresh bacterial suspension were inoculated and spread on the left side of the same clean slide, forming a thin smear. 1-2 loops of the target strain's concentrated bacterial suspension were inoculated and spread on the right side of the another clean slide, forming a thin smear. The thin smears were fixed on the slide by holding the slide with the smear facing up at one end and passing it through a flame 2-3 times. The fixed thin smears were stained by adding an appropriate amount (enough to cover the smear) of crystal violet staining solution for 1 minute and then rinsed gently with water after pouring the staining solution off. The rinsed thin smears were mordanted by adding Lugol's iodine solution for 1 minute and washed with water to rinse off the iodine solution. After tilting the slide, the washed thin smears were decolorized by consecutively dropping 95% ethanol through the smear for 20-25 seconds until the efflux was colorless, and immediately rinsed with water. Then, the smears were redyed by dropping safranin counterstain for 3-5 minutes and the safranin was rinsed off the slide. The stained slides were air-dried.

Observation Under Microscope: The Gram staining reactivity of the bacteria was evaluated using low-magnification, high-magnification, and oil-immersion lenses.

### 2.2 Experimental Results

The results of Gram staining are shown in FIG. 11. The cells of strain BJ-1 were short rod-shaped with blunt-rounded ends, and the stained cells were purple, indicating that it is a Gram-positive bacterium.

### 3. Molecular Identification of Strain BJ-1

### 3.1 Extraction of Genomic DNA

The saved bacterial suspension was spread on a petri dish, and incubated at 25° C. for 3 days. The bacterial cells were scraped off with an inoculation loop and washed out using the LB liquid medium. The genomic DNA was extracted from bacterial cells using the Genomic DNA Rapid Extraction Kit from Beijing Biomed Gene Technology Co., Ltd.

### 3.2 PCR Amplification of 16S rDNA

The genomic DNA was extracted from BJ-1 and its bacterial genomic DNA was used as a template. The universal primers 27f and 1492r for bacterial 16S rDNA were selected and used for PCR amplification.

### Primers for the PCR Amplification:

| | |
|---|---|
| 27f: | 5′-AGAGTTTGATCCTGGCTCAG-3′ |
| 1492r: | 5′-TACCTTGTTACGACTT-3′ |

PCR Amplification Reaction System: 25 μl of 2×Taq PCR Mix, 2 μl of the DNA template (100 ng/μl), 1 μl each of upstream and downstream primers (10 μmol/L), sterile ddH₂O to a final volume of 50 μl.
PCR Reaction Program: 94° C. for 3 min; 94° C. for 30 s, 54° C. for 30 s, 72° C. for 90 s, for 35 cycles; followed by 72° C. for 10 min; stored at 4° C.

The PCR amplification products were detected by electrophoresis, with a fragment length of approximately 1500 bp (SEQ ID NO.: 1 in the sequence listings).

### 3.3. Sequence Analysis and Molecular Identification

After identification of the PCR products by 1% agarose gel electrophoresis, they were sent to Beijing Biomarker Technologies Co., Ltd. for sequencing. The sequencing results of the 16S rDNA PCR amplification fragment of the BJ-1 strain are shown in SEQ ID NO.: 1 in the sequence listings. The obtained sequences were aligned with those in the GenBank database on the NCBI website to identify the species with the highest similarity to the sequenced 16S rDNA sequence. The results showed that the similarity between the 16S rDNA sequence of the BJ-1 strain and that of Bacillus velezensis BCRC 17467T was 99.85%. It was preliminarily identified as a strain closely related to Bacillus velezensis.

Based on the gene fragment sequences, the phylogenetic tree was constructed using the Maximum Likelihood (ML) method based on the 16S rDNA sequences with MEGA software, as shown in FIG. 12. The results indicated that strain BJ-1 clusters with the Bacillus velezensis strain AB78 (GenBank accession number: MN100588.1). Based on this, the strain BJ-1 was identified as Bacillus velezensis. The strain BJ-1 is preserved at the China General Microbiological Culture Collection Center (Address: Institute of Microbiology, Chinese Academy of Sciences, No. 3, Beichen West Road, Chaoyang District, Beijing). The preservation date is Dec. 16, 2021, and the preservation number is CGMCC No. 24113.

### Example 3. Antibacterial Activity Assay of Bacillus velezensis BJ-1

Using the confrontation cultivation method, a total of 59 strains of 26 plant pathogenic fungi (information on plant pathogenic fungi strains is shown in Table 1, and they were identified by the Plant Disease Prevention and Control Research Laboratory of the Beijing Academy of Agriculture and Forestry Sciences using morphological and molecular identification methods and Koch's postulates) were selected as target fungi to determine antagonistic activities of strain BJ-1.

The strain BJ-1 was cultured on LB solid medium at 25° C. for 2-3 days. A single colony was then picked and transferred into 1 mL of LB liquid medium and incubated at 37° C. with shaking 200 rpm/min for 24 h. Fifty-nine pathogenic strains were cultured on PDA plates for 3-5 days. Pathogenic discs with a diameter of 5 mm were prepared from the edge of the colony area by using a hole puncher, and placed on PDA plates. Simultaneously, 4 μl of the cultured bacterial suspensions were pipetted onto filter paper discs with a diameter of 6 mm, which were placed approximately 1 cm away from the edge of the plate, with four discs on each plate. The same volumes of sterile water was pipetted onto filter paper discs as controls, with three replicates. All filter paper discs were incubated at 28° C. for 5 days in a culture chamber and observed for the formation of inhibition zones. The inhibition rate was calculated by measuring the radius (D) of pathogenic hyphae growth opposite to the direction of endophytic antagonistic bacterium BJ-1 and the width of the inhibition zone (d).

$$Inhibition rate ( \% ) = \frac{( colony radius of control - colony radius of treatment )}{colony radius of control} \times 100 \%$$

TABLE 1
Inhibitory activity of strain BJ-1 against 59 plant pathogenic fungal strains.

| Isolated | | Strain | CK Colony | BJ-1 Colony | Inhibition |
|---|---|---|---|---|---|
| Plants | Pathogenic Fungi | Number | Radius (cm) | Radius (cm) | Rate (%) |
| Grape | Botrytis cinerea | SX-Ash | 3.23 ± 0.05 | 1.13 ± 0.05 | 64.95 |
| | | HB-2 | 2.3 ± 0.21 | 0.60 ± 0.08 | 73.91 |
| | Diaporthe eres | HS3-1 | 3.53 ± 0.12 | 1.07 ± 0.05 | 69.81 |
| | | DB-3 | 3.43 ± 0.17 | 0.83 ± 0.05 | 75.73 |
| | Colletotrichum | YT-Carbon | 3 ± 0.08 | 1.03 ± 0.05 | 65.56 |
| | viniferum | BT-2-Carbon | 4.17 ± 0.05 | 1.27 ± 0.05 | 69.45 |
| | Diaporthe sojae | OSS\textcircled{1} | 4.3 ± 0.00 | 0.07 ± 0.00 | 98.45 |
| | | GG-6 | 3.97 ± 0.05 | 0.90 ± 0.08 | 77.31 |
| | Neopestalotiopsis | JL-4-\textcircled{5} | 3.73 ± 0.09 | 1.33 ± 0.05 | 64.29 |
| | sp. | GXXA2-1 | 3.1 ± 0.08 | 1.40 ± 0.08 | 58.06 |
| | | PTP038 | 4.3 ± 0.00 | 1.57 ± 0.05 | 63.57 |
| | Neofusicoccum | SCCDJF-01S | 4.3 ± 0.00 | 1.00 ± 008 | 76.74 |
| | parvum | AH-3-1-01S | 4.3 ± 0.00 | 1.20 ± 0.08 | 72.09 |
| | Coniella vitis | clmsl33-2F | 4.3 ± 0.00 | 1.30 ± 0.08 | 69.77 |
| | | JZB33100014 | 4.3 ± 0.00 | 1.30 ± 0.05 | 72.87 |
| | Lasiodiplodia | CSS-01S | 4.3 ± 0.00 | 1.40 ± 0.08 | 67.44 |
| | theobromae | GX-5 area 5 | 4.3 ± 0.00 | 1.67 ± 0.17 | 61.24 |
| | Botryosphaeria | GXZYD2s | 4.3 ± 0.00 | 1.60 ± 0.08 | 62.79 |
| | dothidea | GS-01S\textcircled{2} | 4.3 ± 0.00 | 1.47 ± 0.12 | 65.89 |
| | Dactylonectria | JZB3310008 | 1.7 ± 0.08 | 0.57 ± 0.05 | 66.67 |
| | macrodidyma | | | | |
| | Lasiodiplodia | WHSB4P03S | 4.3 ± 0.00 | 1.85 ± 0.05 | 57.36 |
| | pseudotheobromae | | | | |
| | Dactylonectria | JZB3310015 | 2.1 ± 0.08 | 0.70 ± 0.08 | 66.67 |
| | torresensis | | | | |
| | Dactylonectria | JZB3310014 | 2.57 ± 0.17 | 0.77 ± 0.09 | 74.03 |
| | alcacerensis | JZB3310013 | 2.77 ± 0.17 | 0.70 ± 0.08 | 74.70 |
| Strawberry | Neopestalotiopsis | 340066 | 3.1 ± 0.14 | 1.43 ± 0.05 | 53.76 |
| | rosae | 340065 | 2.97 ± 0.05 | 1.33 ± 0.09 | 55.06 |
| | Fusarium | LGS15 | 3.6 ± 0.00 | 1.37 ± 0.05 | 62.04 |
| | oxysporum | | | | |
| | Colletotrichum | 330161 | 3.7 ± 0.08 | 1.17 ± 0.09 | 68.47 |
| | siamense | | | | |
| | Colletotrichum | 333003 | 4.3 ± 0.00 | 1.23 ± 0.05 | 71.32 |
| | fructicola | 333002 | 4.3 ± 0.00 | 1.17 ± 0.05 | 72.87 |
| | Colletotrichum | 333001 | 4.3 ± 0.00 | 1.33 ± 0.05 | 68.99 |
| | sojae | | | | |
| Cherry | Alternaria | A1 | 3.57 ± 0.29 | 1.47 ± 0.09 | 58.88 |
| | alternata | A2 | 3.3 ± 0.08 | 1.37 ± 0.17 | 58.59 |
| | Colletotrichum | JZB330210 | 4.17 ± 0.05 | 1.20 ± 0.08 | 71.20 |
| | aenigma | JZB330211 | 3.73 ± 0.05 | 0.80 ± 0.20 | 76.79 |
| | Colletotrichum | JZB330237 | 3.73 ± 0.12 | 1.20 ± 0.08 | 67.86 |
| | gloeosporioides | JZB330239 | 3.67 ± 0.05 | 1.10 ± 0.08 | 70.00 |
| | Colletotrichum | JZB330291 | 4.07 ± 0.21 | 1.27 ± 0.20 | 68.85 |
| | sojae | JZB330293 | 4.23 ± 0.05 | 1.00 ± 0.16 | 76.38 |
| | Botryosphaeria | JZB310198 | 4.3 ± 0.00 | 1.53 ± 0.05 | 64.34 |
| | dothidea | JZB310202 | 4.3 ± 0.00 | 1.57 ± 0.05 | 63.57 |
| | | GZZG7-A-S1 | 4.3 ± 0.00 | 1.47 ± 0.09 | 65.89 |
| | | SYZG-13-A- | 4.3 ± 0.00 | 1.30 ± 0.08 | 69.77 |
| | | S1 | | | |
| | | JZB320189 | 4.3 ± 0.00 | 0.83 ± 0.12 | 80.62 |
| | | B2 | 4.3 ± 0.00 | 0.47 ± 0.05 | 89.15 |
| | | JZB320190 | | | |
| | Cladosporium | TZZG-6-A- | 3.83 ± 0.05 | 0.57 ± 0.05 | 85.22 |
| | cladosporioides | S1 | | | |
| | | TZZG-11-A- | 4.1 ± 0.08 | 0.60 ± 0.08 | 85.37 |
| | | S1 | | | |
| | Diaporthe | GZZG2-S1 | 4.3 ± 0.00 | 1.27 ± 0.05 | 70.54 |
| | honkonggensis | GZZG2-S3 | 4.3 ± 0.00 | 1.30 ± 0.08 | 69.77 |
| | Diaporthe eres | TZZG36-S1 | 4.3 ± 0.00 | 0.83 ± 0.17 | 80.62 |
| | | SDZG35-C- | 4.23 ± 0.05 | 1.10 ± 0.08 | 74.02 |
| | | S2 | | | |
| | Fusarium | Xgld | 3.6 ± 0.00 | 1.23 ± 0.12 | 65.74 |
| | oxysporum | BJTZ-SS-1 | 4.3 ± 0.00 | 1.80 ± 0.08 | 58.14 |
| Tomato | Botrytis cinerea | BJFS-BC-36 | 4.27 ± 0.05 | 1.37 ± 0.05 | 67.97 |
| Eggplant | | BJTZ-BC-1 | 4.3 ± 0.00 | 1.50 ± 0.08 | 65.12 |
| Tomato | Alternaria | BJHD-AL-2 | 4.07 ± 0.05 | 1.17 ± 0.05 | 71.31 |
| Pepper | alternata | BJYH-CG-3 | 3.37 ± 0.05 | 1.33 ± 0.05 | 60.40 |

By confrontation cultivation method, the inhibitory activities of strain BJ-1 against 59 strains of 26 plant pathogenic fungi were measured, and the test results are shown in Table 1. The experimental results showed that the strain BJ-1 had significant prevention and control effects on the tested different plant pathogenic fungi, with the inhibition rates of mycelial growth ranging from 53.76% to 98.45%. The strain BJ-1 achieved the most effective inhibition of mycelial growth of Diaporthe sojae OSS (1), which causes the grapevine canker disease, with an inhibition rate of up to 98.45%. The study results indicated that BJ-1 had broad-spectrum prevention and control effects on common fruit and vegetable plant pathogenic fungi.

### Example 4. Detached Leaf Disc Inoculation Method to Determine Protection and Control Effects of the Strain BJ-1 on Tomato Gray Mold

### 1. Experiment Method

### 1.1. Pathogen Preparation

Tomato gray mold fungus (Botrytis cinerea) strains were preserved by the Phytopathology Research Laboratory of Beijing Academy of Agricultural and Forestry Sciences, and identified using routine methods. The preserved strains were inoculated onto PDA plates and cultured in an incubator at 25° C.

### 1.2. Agent Preparation

The biocontrol bacterium BJ-1 powder was formulated with sterile water as a solvent into four concentration gradients of 1×10⁵ spores/mL, 1×10⁶ spores/mL, 5×10⁶ spores/mL, and 1×10⁷ spores/mL for later use.

### 1.3. Experiment Treatments

### 1.3.1. Determination of Protection Effects of Strain BJ-1 on Tomato Gray Mold

The drug treatment was carried out by a spray method, including spraying the bacterial suspension evenly on the back of tomato leaves with a throat sprayer nozzle until the suspension just started to flow down the leaves. After 24 hours, the cakes of tomato gray mold fungus (4 mm in diameter) were inoculated, with the side containing hyphae facing down and aligned with the inoculation point on the leaves. The petioles were wrapped with moistened, grease-free cotton, and then placed in a fresh-keeping box with double-layer filter paper at the bottom for humidification. They were incubated at 25° C. for 3-5 days before determining the results. Each repetition included 6-8 leaves, with three repetitions. Water and a control drug (100 billion CFU/g Bacillus subtilis wettable powder) were used as controls.

### 1.3.2. Control Effect of Strain BJ-1 on Tomato Gray Mold

Control effect was determined by first inoculating the pathogen, and 24 hours post-inoculation, treating tomato leaves with different bacterial suspensions or sterile water. The methods and materials used were identical to those for employed in the protection effect determination.

### 1.4. Data Statistical Analysis

The cross method was used to measure the diameters of disease spots on each of treated fruits. The relative prevention and control effects were calculated according to the formula.

$$Prevention And Control Effects ( \% ) = \frac{( Diameter Of Disease Spot Of Control - Diameter Of Cake ) - ( D i a meter Of Disease Spot Of Treatment - Diameter Of Cake )}{Diameter Of Disease Spot Of Control - Diameter Of Cake} \times 100 \%$$

### 2. Experimental Results

Through detached leaf inoculation, the prevention and control effects of the strain BJ-1 on tomato gray mold were evaluated, and the results are shown in Table 2. The results indicated that the suspensions of strain BJ-1 at four concentrations had certain protection effects on tomato gray mold. With increasing concentration, the prevention and control effects gradually improved. Among them, the fermentation liquid of BJ-1 at a concentration of 1×10⁷ CFU/mL showed the best prevention and control effects, reaching 68.95% and 88.28%, respectively. The prevention and control effects on tomato gray mold for 10⁹ CFU/g Bacillus subtilis wettable powder (Bayer CropScience, pesticide registration number: PD20160669) as the control, were 49.19% and 20.08%, and significantly lower than those of the strain BJ-1. Therefore, the prevention and control effects of the strain BJ-1 on tomato gray mold are significantly better than those of 10⁹ CFU/g Bacillus subtilis wettable powder as the control.

TABLE 2
Prevention and control effects of strain BJ-1 on tomato
gray mold (in vitro inoculation on detached leaves)

| | Protection effect | | Control effect | |
|---|---|---|---|---|
| | | Prevention and | | Prevention and |
| | Lesion length | control effect | Lesion length | control effect |
| Treatments | (cm) | (%) | (cm) | (%) |
| BJ-1 1 × 10⁵ CFU/mL | 1.11 ± 0.23 | 19.35 | 0.78 ± 0.08 | 41.18 |
| BJ-1 1 × 10⁶ CFU/mL | 0.68 ± 0.19 | 50.40 | 0.36 ± 0.02 | 73.22 |
| BJ-1 5 × 10⁶ CFU/mL | 0.65 ± 0.09 | 52.82 | 0.16 ± 0.03 | 87.87 |
| BJ-1 1 × 10⁷ CFU/mL | 0.43 ± 0.15 | 68.95 | 0.15 ± 0.04 | 88.28 |
| 10 Billion CFU/mL | 0.70 ± 0.23 | 49.19 | 1.06 ± 0.08 | 20.08 |
| Bacillus subtilis WP | | | | |
| 1 × 10⁸ CFU/mL | | | | |
| Sterile water | 1.38 ± 0.04 | — | 1.33 ± 0.03 | — |

### Example 5. Field Prevention and Control Effects of 2.0×10¹⁰ CFU/g Bacillus velezensis BJ-1 Water-Dispersible Powder on Tomato Gray Mold

### 1. Materials and Methods

### 1.1. Tested Drug

The tested drug was water-dispersible powders of Bacillus velezensis BJ-1, containing 2.0×10¹⁰ CFU/g (The final concentration of BJ-1 powder was 2.0×10¹⁰ CFU/g; polyvinyl alcohol 6.0%; sodium dodecyl sulfate 4.0%; ammonium sulfate 4%; polyethylene glycol 4%; and talcum powder supplemented to 100%. The percentage content was on a mass basis). The control drug is Bacillus subtilis wettable powder, containing 100 billion spores/g (Jiangxi Zhengbang Crop Protection Co., Ltd., pesticide registration number: PD20151587).

### 1.2. Experimental Plant Materials

The experimental crop was tomato, “Jingcai 8”, with a growth period from flowering to fruiting.

### 1.3. Experiment Methods

The information on treatments with the drugs is presented in Table 3, with each plot area of 20 m² and four repetitions for each of the treatments. Spraying was conducted during the early stage of tomato gray mold disease. The first spray occurred on Dec. 29, 2022, followed by subsequent sprays every 7 days for a total of 3 times, and the investigation on the disease prevention and control effects was conducted on Jan. 20, 2023. The investigation followed the standards of the People's Republic of China agricultural industry, “Guidelines for Pesticide Field Efficacy Trials (1): bactericides for the Prevention and Control of Gray Mold of Vegetables” (GB/T 17980.28-2000).

TABLE 3
Experiment designs of tested drugs.

| Treatment | | Dosage |
|---|---|---|
| No. | Drugs | (g/mu) |
| 1 | 2.0 × 10¹⁰ CFU/kg Bacillus velezensis | 112.5 |
| 2 | BJ-1 water-dispersible powder | 225 |
| 3 | | 450 |
| 4 | 1.0 × 10¹² spores/kg Bacillus subtilis | 70 |
| | wettable powder | |
| 5 | Control | Water |

The disease index and the prevention and control effects were calculated using the following formula.

$$Illness index = \frac{\sum ( Number of diseased leaves at all levels - Relative level value )}{Total number of leaves investigated \times 9} \times 100$$

$$Prevention And Control Effects ( \% ) = \frac{P o s t - drug Illness index of control area - P o s t - drug Illness index of treatment area}{P o s t - drug Illness index of control area} \times 100$$

### 1.4. Data Statistical Analysis

Statistical analysis of the experimental data was conducted using SPSS 25.0 software. A difference significance test of the experiment result data was conducted using Duncan's new multiple range test (DMRT).

### 2. Results

According to the results of field investigation, 2.0×10¹⁰ CFU/g Bacillus velezensis BJ-1 water-dispersible powder, as the tested drug, had good prevention and control effects on tomato gray mold, with average prevention and control effects ranging from 72.38% to 80.48%. The post-treatment average prevention and control effects of treatment 1 (112.5 g/acre), treatment 2 (225 g/acre), treatment 3 (450 g/acre) and 100 billion spores/g B. subtilis wettable powder (70 g/acre) as a control were 72.38%, 76.05%, 80.48%, and 66.48% respectively.

The variance analysis results indicated that, at the 0.05 level, there were significant differences in the prevention and control effects on tomato gray mold between treatment 1 (112.5 g/ha) using 2.0×10¹⁰ CFU/g Bacillus velezensis BJ-1 water-dispersible powder, treatment 2 (225 g/ha), treatment 3 (450 g/ha) and 100 billion spores/g Bacillus subtilis wettable powder (70 g/ha) as the control. At the 0.01 level, there were extremely significant differences in prevention and control effects between treatment 1 (112.5 g/ha) using 2.0×10¹⁰ CFU/g Bacillus velezensis BJ-1 water-dispersible powder, treatment 2 (225 g/ha), and treatment 3 (450 g/ha) and 100 billion spores/g Bacillus subtilis wettable powder as the control (70 g/ha). Therefore, 2.0×10¹⁰ CFU/g Bacillus velezensis BJ-1 water-dispersible powder can be used as a drug to prevent and control tomato gray mold. It is recommended to spray the formulation before or at the onset of the disease, at a dosage of 225-450 g/ha.

The above-described embodiments are merely illustrative of the present invention, which is described in detail but is only indicative rather than restrictive. Ordinary skilled persons in the art can make many modifications, changes, and improvements, without departing from the spirit and scope defined by the appended claims, all of which are within the scope of the present invention.

1. A strain of Bacillus velezensis, wherein the strain is named Bacillus velezensis BJ-1, and deposited in China General Microbiological Culture Collection Center (Address: No. 3, Courtyard No. 1, Beichenxi Road, Chaoyang District, Beijing) on Dec. 16, 2021 with a depository number CGMCC No. 24113.

2. A use of the strain of Bacillus velezensis according to claim 1 in manufacturing a biocontrol agent, a microbial fertilizer, or a biopesticide that exhibits inhibition of plant pathogenic fungi.

3. The use according to claim 2, wherein the plant pathogenic fungi is selected from any one or more of:
Lasiodiplodia theobromae, Botryosphaeria dothidea, Neofusicoccum parvum, Diaporthe sojae, Diaporthe eres, Diaporthe honkonggensis, Botrytis cinerea, Colletotrichum viniferum, Colletotrichum sojae, Colletotrichum aenigma, Colletotrichum gloeosporioides, Colletotrichum fructicola, Colletotrichum siamense, Colletotrichum acutatum, Neopestalotiopsis sp., Neopestalotiopsis Rosae, Coniella vitis, Dactylonectria alcacerensis, Dactylonectria macrodidyma, Lasiodiplodiapseudotheobromae, Dactylonectria torresensis, Fusarium oxysporum, anastomosis group A of binucleate Rhizoctonia, anastomosis group G of binucleate Rhizoctonia, Alternaria alternata, Cladosporium cladosporioides, and Sclerotinia sclerotiorum.

4. A biocontrol agent for plant pathogens, wherein an active ingredient of the biocontrol agent is the strain of Bacillus velezensis according to claim 1.

5. The biocontrol agent according to claim 4, wherein the plant pathogenic fungi is selected from any one or more of:
Lasiodiplodia theobromae, Botryosphaeria dothidea, Neofusicoccum parvum, Diaporthe sojae, Diaporthe eres, Diaporthe honkonggensis, Botrytis cinerea, Colletotrichum viniferum, Colletotrichum sojae, Colletotrichum aenigma, Colletotrichum gloeosporioides, Colletotrichum fructicola, Colletotrichum siamense, Colletotrichum acutatum, Neopestalotiopsis sp., Neopestalotiopsis Rosae, Coniella vitis, Dactylonectria alcacerensis, Dactylonectria macrodidyma, Lasiodiplodiapseudotheobromae, Dactylonectria torresensis, Fusarium oxysporum, anastomosis group A of binucleate Rhizoctonia, anastomosis group G of binucleate Rhizoctonia, Alternaria alternata, Cladosporium cladosporioides, and Sclerotinia sclerotiorum.

6. A microbial fertilizer for plant diseases, wherein an active ingredient of the microbial fertilizer is the strain of Bacillus velezensis according to claim 1.

7. The microbial fertilizer according to claim 6, wherein the plant pathogenic fungi is selected from any one or more of:
Lasiodiplodia theobromae, Botryosphaeria dothidea, Neofusicoccum parvum, Diaporthe sojae, Diaporthe eres, Diaporthe honkonggensis, Botrytis cinerea, Colletotrichum viniferum, Colletotrichum sojae, Colletotrichum aenigma, Colletotrichum gloeosporioides, Colletotrichum fructicola, Colletotrichum siamense, Colletotrichum acutatum, Neopestalotiopsis sp., Neopestalotiopsis Rosae, Coniella vitis, Dactylonectria alcacerensis, Dactylonectria macrodidyma, Lasiodiplodiapseudotheobromae, Dactylonectria torresensis, Fusarium oxysporum, anastomosis group A of binucleate Rhizoctonia, anastomosis group G of binucleate Rhizoctonia, Alternaria alternata, Cladosporium cladosporioides, and Sclerotinia sclerotiorum.

8. A biopesticide for plant diseases, wherein an active ingredient of the microbial fertilizer is the strain of Bacillus velezensis according to claim 1.

9. The biopesticide according to claim 8, wherein the biopesticide comprises excipients and/or auxiliaries acceptable to a pesticide formulation.

10. The biopesticide according to claim 8, wherein the biopesticide is a form of water-dispersible powders, containing 1.0×10⁸ CFU/g or more Bacillus velezensis BJ-1 which is the strain of Bacillus velezensis according to claim 1.

11. The biopesticide according to claim 10, wherein the water-dispersible powder is composed of: Bacillus velezensis BJ-1 at a final concentration of 1.0×10⁸-1.0×10¹¹, 6.0% polyvinyl alcohol, 4.0% sodium dodecyl sulfate, 4% ammonium sulfate, 4% polyethylene glycol; and talc powder supplemented to 100%; and the percentage content is a mass percentage.


SEQUENCE LISTING (1 sequence)

SEQ ID NO: 1 - DNA, 1444 bp, Bacillus velezensis
  source 1..1444: mol_type = genomic DNA
gacgtaccttcggcggctggctcataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattccttggagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcaaacttaagaaaccgcctgcgaaccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcagacaaccatccggtattagccccggtttcccggagttatcccagtcttacaggcaggttacccacgtgttactcacccgtccgccgctaacatcagggagcaagctcccatctgtccgctcgacttgcagtatagcactcgtcg